(19) United States
(12) Patent Application Publication
(10) Pub. No.: US 2011/0152941 A1
(43) Pub. Date: Jun. 23, 2011

(54) EXTRA-DISCAL ASSEMBLY FOR INTERVERTERBRAL STABILISATION FOR ARTHRODESIS

(76) Inventor: Henry Graf, Lyon (FR)

(21) Appl. No.: 13/058,817

(22) PCT Filed: Jul. 17, 2009
(86) PCT No.: PCT/FR09/00880

§ 371 Date: Feb. 11, 2011

(30) Foreign Application Priority Data
Aug. 14, 2008 (FR) 08 55574

(51) Int. Cl. A61B 17/70; A61B 17/88

(52) U.S. Cl. 606/277; 606/279

(57) ABSTRACT

The invention relates to an assembly that includes: at least two rods (10, 20) capable of interacting with at least two different vertebrae; at least two pairs of jaws (50, 60, 70, 80), each pair of jaws being arranged in the vicinity of a corresponding rod; at least two slides (30, 40) each defining a longitudinal axis of said assembly, and at least one jaw from each pair of jaws being capable of sliding along at least two slides along the longitudinal axis thereof; a return means (74, 84) for each pair of jaws in contact with a corresponding vertebral rod, each vertebral rod being capable of moving at least one jaw along the slides towards the return means, and each vertebral rod further being articulated relative to each jaw.

The present invention relates to an extra-discal assembly for intervertebral stabilization for arthrodesis.

The invention lies in the field of arthrodesis, i.e. bone fusion between at least two adjacent vertebrae. It is recalled that arthrodesis sets out to allow only micromovements between the vertebrae, and also to damp vibration. Such micromovements enable patients, who can once again adopt an upright posture after the operation, to adapt their equilibrium as well as possible before the bone graft takes. These micromovements also make it possible, after bone fusion has occurred, for the assembly in accordance with the invention to avoid opposing plastic adaptations, which are one of the fundamental characteristics of variation in the vertebral column during the lifetime of the patient.

In typical manner, an extra-discal assembly for arthrodesis in the meaning of the invention allows movement between two vertebrae to have an amplitude, in side view, that is equal to no more than about 10% of the natural physiological amplitude. In other words, if a natural maximum amplitude in pivoting between two given adjacent vertebrae exists with a value of 10°, then the stabilization assembly in accordance with the invention is suitable for allowing those two vertebrae to move but through no more than 1°.

It should be observed that it is fundamental to make a distinction between firstly a prosthesis designed to recreate intervertebral movement, and secondly an assembly of the invention having the sole purpose of obtaining bone fusion between two vertebrae. In this respect, it should be recalled that there exist substantial structural and functional differences between a prosthesis and an osteosynthesis device, such that a prosthesis cannot allow bone fusion to take place and, in similar manner, an arthrodesis assembly cannot perform a prosthetic function.

The stabilization assembly in accordance with the invention is for connecting together at least two adjacent vertebrae while generally being placed on one side only of the vertebral column, i.e. on the left or the right. This stabilization element is implanted in an extra-discal manner, i.e. it may be situated behind, or alternatively in front of, the vertebral space.

A stabilization assembly is already known, such as that made available by the supplier Medtronic under the trade reference Agile. That assembly comprises two rigid members suitable for co-operating with two pedicular screws implanted in two adjacent vertebrae. A damping buffer is also provided that is fitted against the end plates of the rigid members, in particular by hot vulcanization. Under such conditions, the buffer may not only be compressed, while remaining secured to those two members, it may also be stretched, because of the presence of the bonding.

That Agile stabilization assembly also includes centering means for preventing the two rigid members from moving off axis, in particular during flexion movements. For this purpose, use is made of a cable that is secured to one of the two rigid members, that passes through the damping buffer, and that extends into the inside volume of the facing hollow rigid member.

That known solution nevertheless presents certain drawbacks. In particular, the Agile stabilization assembly tends to work much too much in flexion, thereby reducing its effectiveness and its lifetime. It should also be observed that that assembly, which was initially designed as a prosthesis for accompanying vertebral movement, was transformed into an osteosythesis device as a result of administrative constraints in the United States. Such a transformation was implemented without structurally modifying the assembly so as to restrict its movement, thereby showing that that assembly cannot obtain arthrodesis since it allows movements to take place to much too great an extent.

Stabilization assemblies are also known that are sold under the references Dynesis and Nflex. Those posterior dynamic stabilization systems make use of the resilient mechanical properties of elastomer buffers to limit the mobility of pedicular screws during intervertebral movement. Under such conditions, the movement of each screw is the result of deforming one of those buffers in compression, for flexion if the buffer is on the outside of the space between the screws, or for extension if the buffer is situated between the screws.

The main drawback of those known devices lies in the absence of articulation between the screw and the buffer. As a result, when the screw is caused to pivot in the context of a flexion-extension movement, it causes the buffer to perform bending work. Under such conditions, the buffer stressed in that way in turn causes the screw to pivot, and that does not lie within physiologically natural intervertebral movement.

Furthermore, if it is desired to approach physiologically natural movement by means of those two devices, it is necessary for the stiffness of the buffers to increase very considerably. Under such conditions, those devices approximate to rigid systems, thereby giving rise to consequences concerning stresses on the implanted screws, and also giving rise to insufficient absorption of impacts and vibration. The buffers thus behave more like abutments than like dampers, with any residual movement that is obtained resulting solely from the flexibility of the system.

That said, the invention seeks to remedy those various drawbacks. To this end, the invention provides an extra-discal assembly for intervertebral stabilization for arthrodesis, the assembly comprising:
at least two rods referred to as “vertebral” rods, suitable for co-operating with at least two different vertebrae;
at least two pairs of jaws, each pair of jaws being placed in the vicinity of a corresponding rod;
at least two slideways defining a longitudinal axis of said assembly, at least one jaw in each pair of jaws being suitable for sliding along at least two slideways, along their respective longitudinal axes; and
return means for returning at least one jaw in each pair of jaws towards a corresponding vertebral rod; each vertebral rod being suitable, in operation, for moving at least one jaw along the slideways against the return means, each vertebral rod also being articulated relative to each jaw.

According to other characteristics:
Said articulation between the rod and each jaw acts via a single contact point between said rod and the facing wall of each jaw.
At least one jaw comprises a curved connection branch having a radius of curvature greater than the radius of curvature of the rod.
The curved branch is secured to two tabs mounted on the two slideways, optionally in slidable manner.
The return means comprise two springs, each of which is mounted on a corresponding slideway, these two springs being suitable for returning a curved branch towards the vertebral rod.
At least one jaw includes an articulation stud engaged against a corresponding vertebral rod.
The return means comprise a solid flexible body and the stud forms part of a rigid endpiece secured to the flexible body.
At least one pair of jaws comprises a first jaw slidable along the slideways against the return means, together with a second jaw mounted stationary on the slideways.
At least one pair of jaws comprises first and second jaws mounted slidable along the slideways, with the return means comprising first and second return means, each movable jaw being suitable for sliding against corresponding return means.
The first and second return means are merged into a single return means comprising a central return member between two endpieces, each defining a first respective jaw, two pairs of slideways are provided, each pair defining a respective second jaw, and each pair of slideways is suitable for sliding relative to the endpiece adjacent to the jaw defined by said slideways, said pair of slideways in contrast being secured to move in translation with the opposite endpiece, at least in the direction in which the two endpieces approach each other.
The central return member is a helical spring.
Each rod forms part of a corresponding vertebral screw, in particular a pedicular screw.
Each rod is secured to a connection element suitable for connecting together two vertebral screws for implanting in a common vertebral stage.
The assembly includes stroke-limitation means for limiting the stroke between two adjacent rods.
The stroke-limitation means are means for limiting the compression of a deformable body, forming part of the return means.
The compression-limitation means comprise a rigid chamber extending at a distance from walls of the deformable body when the deformable body is in a rest position.
The compression-limitation means comprise at least one ring surrounding a portion of the deformable body so as to limit its deformation zone;
The assembly includes means serving to prevent the slideways and the jaws sliding along the rods, along the main axes thereof, in at least one direction.
Two slideways are suitable for guiding the rods, in particular by co-operating with respective zones of said rods presenting a cross-section that corresponds substantially to the distance between the slideways.

The invention is described below with reference to the accompanying drawings given purely by way of non-limiting example, and in which:

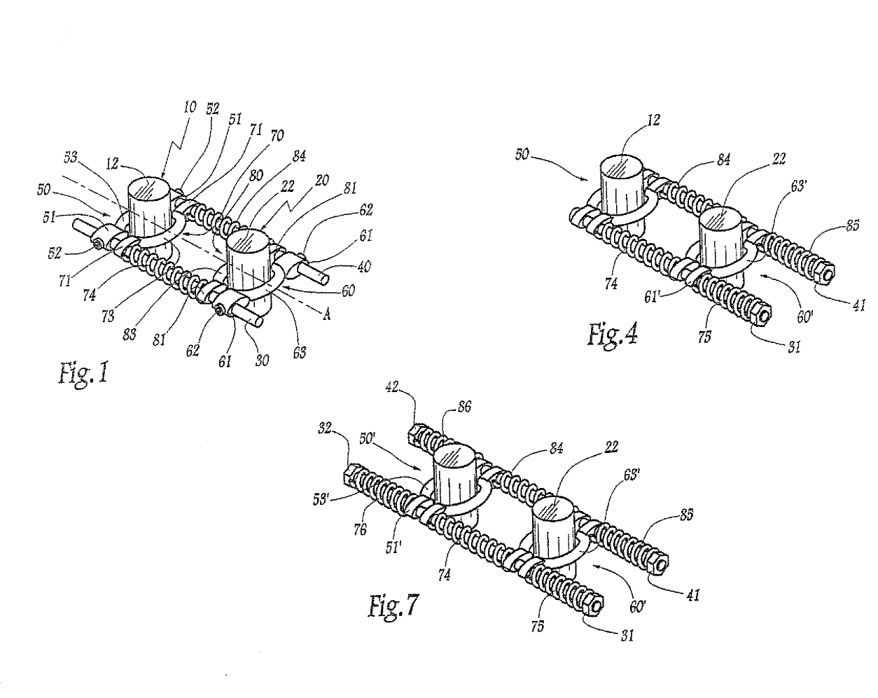

FIG. 1 is a perspective view showing a stabilization assembly in accordance with a first embodiment of the invention;

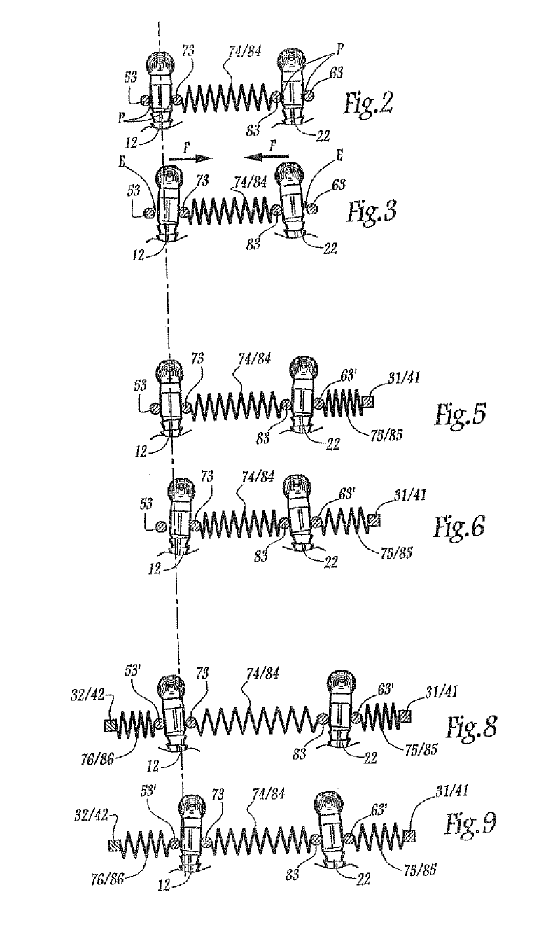

FIGS. 2 and 3 are side views showing how the FIG. 1 assembly is implemented;

FIGS. 4 and 7 are perspective views analogous to FIG. 1, respectively showing two variant embodiments of the invention;

FIGS. 5 and 6 and also 8 and 9 are side views analogous to FIGS. 2 and 3, showing how the assemblies of FIGS. 4 and 7 respectively are implemented;

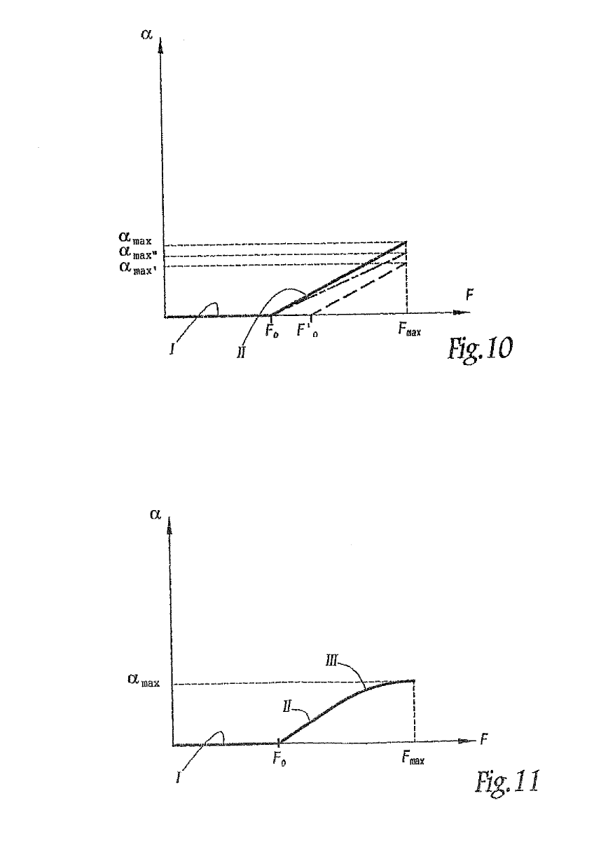

FIG. 10 is a graph plotting variation in the intervertebral angle of inclination as a function of the force applied by the patient in the various embodiments of the invention;

FIG. 11 is a graph, analogous to FIG. 10, plotting said variation in another implementation of the invention;

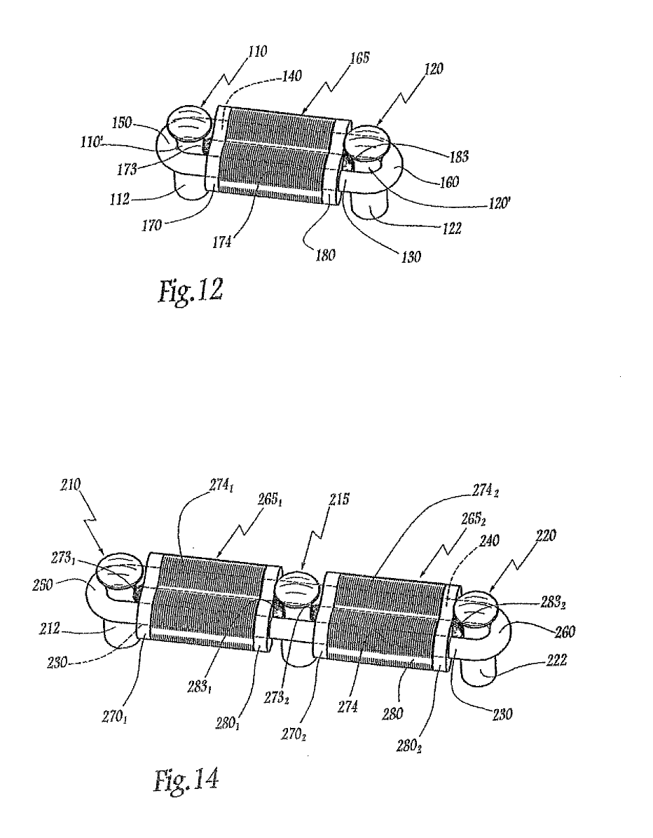

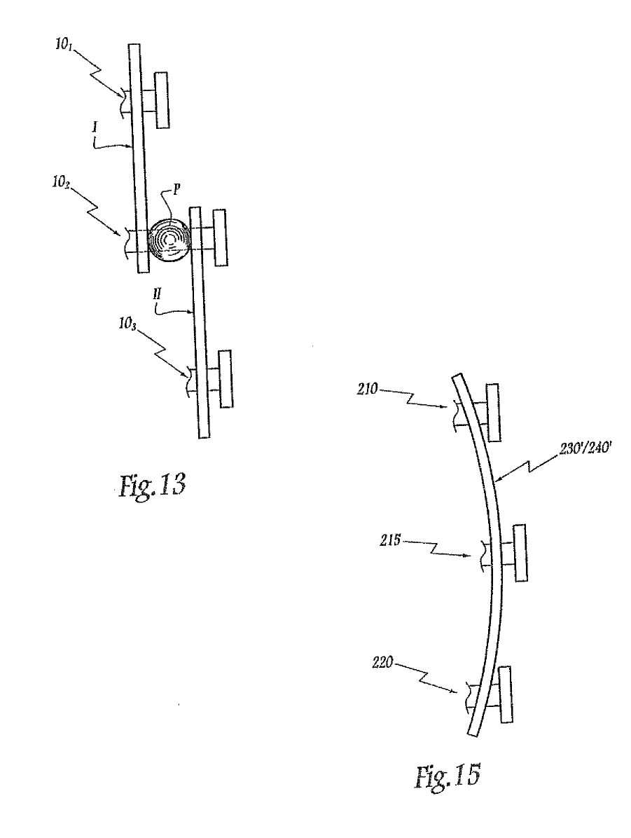

FIGS. 12 and 13 are respectively a perspective view and a side view showing an additional embodiment of the stabilization assembly in accordance with the invention;

FIG. 14 is a perspective view showing a stabilization assembly in accordance with an additional variant embodiment of the invention;

FIG. 15 is a side view showing a variant embodiment of the FIG. 14 assembly;

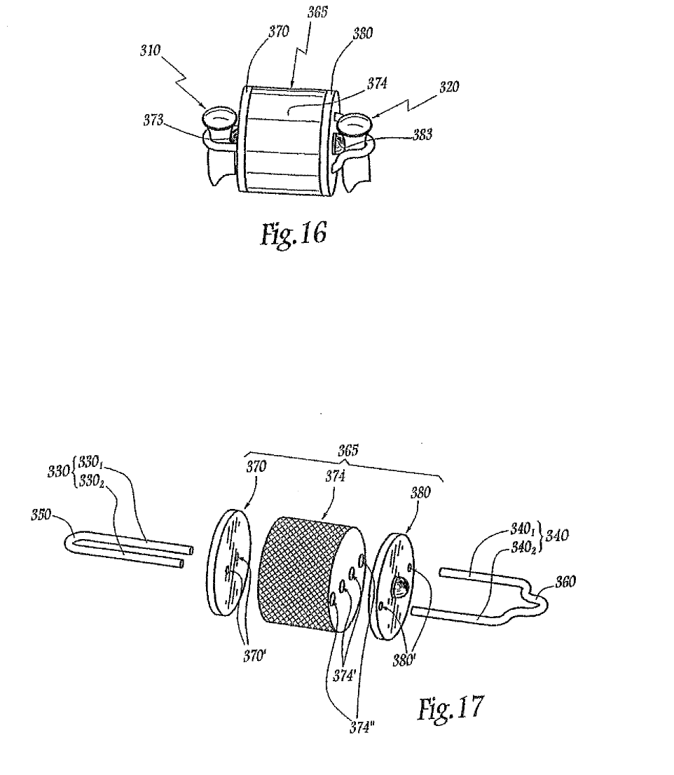

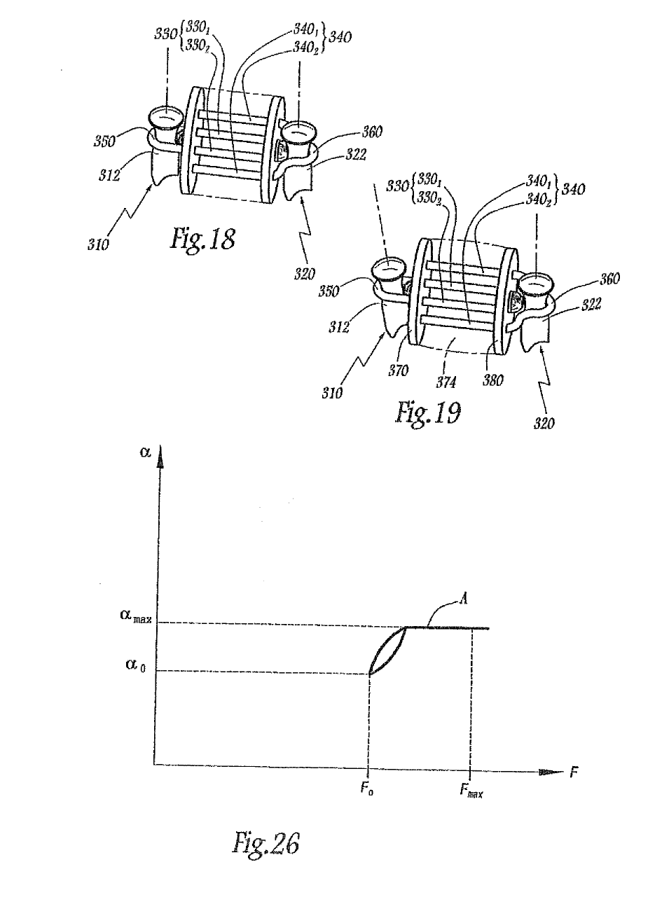

FIGS. 16, 17, and 18 are perspective views showing an extra-discal assembly in accordance with an additional variant of the invention;

FIG. 19 is a perspective view showing an implementation of the assembly of FIGS. 16 to 18;

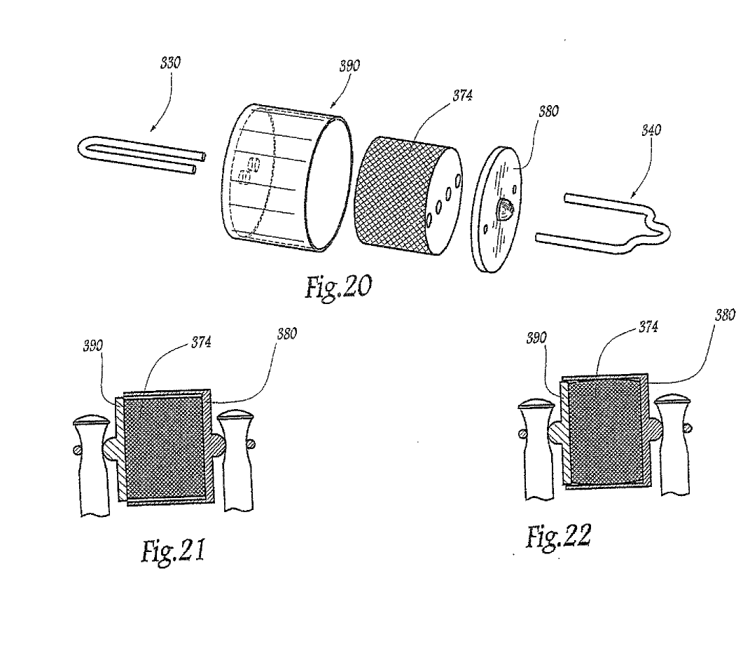

FIG. 20 is an exploded perspective view showing various component elements of an additional variant embodiment of the invention;

FIGS. 21 and 22 are longitudinal section views showing the FIG. 20 assembly in two utilization positions;

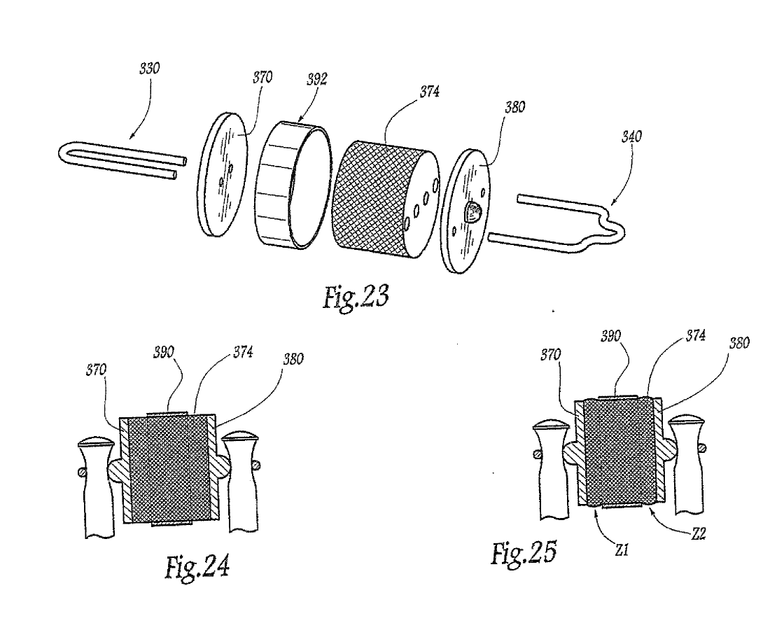

FIGS. 23, 24, and 25 are respectively an exploded perspective view, and longitudinal section views, analogous to FIGS. 20 to 22, showing an additional variant embodiment of the invention; and

FIG. 26 is a graph analogous to FIG. 10 plotting variation in the intervertebral angle of inclination as a function of the applied force for the embodiments of FIGS. 20 to 22.

The extra-discal assembly in accordance with the invention as shown in FIG. 1 comprises firstly two vertebral screws, which are shown in part. These two screws, given respective references 10 and 20 comprise respective cylindrical shanks 12 and 22 that can be seen more clearly in FIG. 1. By way of example, these screws are pedicular screws including, in conventional manner, a threaded zone for penetrating into the vertebral body. Nevertheless, provision may be made to use, not a particular type of screw, but rather any type of vertebral screw.

Thus, the screw may be implanted in the vertebral body, either laterally, or anteriorly, or in the vertebral body through the pedicle. In general, it is possible to provide for any insertion that ensures that the screw is secured stably relative to the vertebra. It is then implanted in the vertebra by a screw thread and it causes a stud to project out from the vertebra suitable for co-operating with a jaw, as described below. The stud may also be supported by a mechanical member other than a thread, e.g. such as a staple or hooks placed on the vertebral body and/or intervertebral bone plates.

The stabilization assembly in accordance with the invention further includes two slideways 30 and 40 extending substantially along the axis A interconnecting the two screws 10 and 20, once they have been implanted in vertebrae. Each slideway is constituted by a corresponding rod 30, 40 that may be rigid, or that may present the ability to deform a little, in the flexion direction. In any event, if it can deform, its ability to deform is controlled. When seen from the side, and regardless of whether or not it is rigid, each rod may be straight, or it may present a little curvature, so as to match the curvature between vertebrae, where appropriate.

Each screw 10 or 20 is associated with two jaws, one of which is stationary and the other of which is mounted to slide relative to the two rods 30 and 40. The two stationary jaws are referenced 50 and 60, and the two movable jaws are referenced 70 and 80.

All four jaws present the same structure, i.e. each of them comprises two tabs 51, 61, 71, 81 for attaching to the corresponding rod. The tabs 51 and 61 of the stationary jaws are provided with respective screws 52, 62 that enable them to be secured relative to the two rods 30 and 40.

Furthermore, the tabs in each facing pair are connected together via respective connection branches 53, 63, 73, and 83. Each connection branch is curved when seen from above, i.e. about the axis of the corresponding screw, so as to make articulated contact with the shank of a corresponding screw. Seen from above, each screw presents a radius of curvature corresponding to its diameter, whereas each curved branch presents a radius of curvature that is greater than the diameter of the screw, thereby making this articulation possible. Furthermore, as shown in FIG. 2, each branch is circular in cross-section, thus also enabling the jaw to be articulated relative to the cylindrical shank of the screw.

Each branch may be rigid, or it may be capable of deforming, at least in some places, under the effect of stresses of magnitude significantly greater than gravity. In contrast, all of the branches present their own shape, i.e. they are of a shape that does not vary under the effect of gravity, nor indeed under the effect of other stresses of analogous magnitude.

It should also be observed that the two movable jaws 70 and 80 are adjacent, i.e. they are disposed on the facing sides of the two shanks. In contrast, the two stationary jaws are further apart from each other, i.e. they are presented against the opposite faces of those two shanks 12 and 22.

Two springs 74 and 84 are interposed between the two facing movable jaws 70 and 80. These springs tend to urge each of the movable jaws 70 or 80 towards the stationary jaw 50 or 60 that is associated therewith.

It should be observed that the greater the extent to which these springs are stiff and/or prestressed, the more they tend to oppose any movement of the movable jaws. The distinction between these respective concepts of stiffness and of prestress is explained in greater detail below. In the context of the present invention, i.e. arthrodesis, the springs used are thus of considerable stiffness and/or prestress, so as to allow only micromovements, and vibration damping, as defined above.

Each shank 12 or 22 is circular in cross-section. In addition, each branch is also circular, likewise in cross-section. Finally, as mentioned above, the branches present a curved profile, i.e. the two facing branches define a shape that is more or less oval, with a radius of curvature that is greater than the radius of the circular shank.

Under such conditions, each shank 12 or 22 co-operates with each branch 53, 73, 63, or 83 to define articulation substantially about a point, as represented by the points P in FIG. 2. In other words, there exist three degrees of freedom in rotation between each branch and the shank with which it co-operates. In FIG. 2, the springs 74 and 84 are shown diagrammatically. In a variant that is not shown, the articulation may be implemented by means of contact that is not a point contact, being a contact of the flat-on-flat type. These two flats, one forming part of the shank and the other forming part of the branch, thus define a contact zone of relatively small area that makes subluxation possible, which is considered as coming within the ambit of “articulation” in the meaning of the present invention.

There follows a description of the operation of the above-described stabilization assembly. It is assumed that the two screws 10 and 20 are pedicular screws and that the top portions of these screws, as seen in FIG. 1, point backward from the patient when the patient is standing.

The jaws are placed first around the screws 10 and 20. In the present example, it is assumed that the stabilization assembly in accordance with the invention is to form a stay. Under such conditions, and in the absence of stress, the free ends of the two screws are spaced apart by a distance that is greater than the distance between the screws once they are associated with the jaws.

In other words, for assembly purposes, the two screws need to be moved manually towards each other, e.g. by means of a tool that is not shown. Thereafter the two jaws together with the two rods are moved axially towards the screws so as to insert the two shanks 12 and 22 through the two eyelets as defined by the jaws. Thereafter the external action exerted by the tool is released so that the shanks 12 and 22 come to bear against the stationary jaws 50 and 60.

If the patient seeks to exert intervertebral flexion, i.e. to lean forwards, then the two screws 10 and 20 tend to move apart from each other along the axis A. However, this is made impossible since the two shanks 12 and 22 then come into abutment against the stationary jaws 50 and 60. The stabilization assembly of the invention thus substantially prevents any intervertebral flexion movement.

In contrast, during intervertebral extension, the screws 10 and 20 tend to move towards each other along the axis A. Unlike flexion, this relative movement of the screws is possible insofar as they then push the movable jaws 70 and 80 towards each other against the springs 74 and 84. The movable jaws consequently tend to slide along the rods 30 and 40.

This is shown more particularly in FIG. 3, where there can be seen the four branches, the two shanks, and the springs 74 and 84. It should be observed that during such intervertebral extension, the shanks 12 and 22 move not only in pivoting, but also in translation along the arrows F, thereby pushing the branches 73 and 83 against the springs 74 and 84. However, given that the jaws 53 and 63 are stationary, they do not move, thereby creating two empty spaces E between the facing walls of said jaws and the facing shanks. Thereafter, when the patient ceases this extension movement, or when the force exerted in this way is less than the stiffness of the springs 74 and 84, the springs push the shanks 12 and 22 back into their original positions, as shown in FIG. 2.

FIG. 4 shows a variant embodiment of the invention. In this figure, those mechanical elements that differ from the corresponding elements in FIGS. 1 to 3 are designates by the same numbers together with a “prime” sign.

In this variant embodiment, the jaw 60′ is no longer stationary, like the jaw 60 in the first embodiment, but on the contrary it is free to slide along the rods 30 and 40. This sliding takes place against two additional springs 75 and 85 interposed between the tabs 61′ of the jaw 60′ and abutments 31 and 41 that are mounted in stationary manner on the slideways 30 and 40.

The jaws and screws forming part of the assembly in accordance with the second embodiment of the invention are mounted in a manner analogous to that described with reference to the first embodiment. Once the shanks 12 and 22 have been inserted in the eyelets defined by the various jaws, the shanks bear against the respective jaws 50 and 60′.

Given that the jaw 60′ is now movable, equilibrium is established between the forces exerted respectively by the screws 20, the “inner” springs 74 and 84, and the “outer” springs 75 and 85. More precisely, the screw 20 exerts a force tending to push it away the first screw 10, i.e. a reaction against the action of the surgeon tending to move them towards each other. In addition, the springs 74 and 84 also tend to push the screw 20 away from the screw 10. In contrast, the springs 75 and 85 tend to urge the screw 20 towards the first screw 10.

In this second embodiment, intervertebral flexion is now possible. Thus, when the patient leans forwards, the branch 53 of the stationary jaw holds the screw 10 stationary, while the screw 20 pushes back the branch 63′ of the movable jaw 60′ against the two springs 75 and 85. Simultaneously, the springs 74 and 84 tend to push back the branch 83 of the jaws 80 towards the shank 22 of the screw 20, as shown in FIG. 5.

In order to explain further the way in which this intervertebral flexion takes place, FIG. 10 plots a curve showing the variations in the angle α as a function of the force F. More precisely, F corresponds to the flexion force exerted by the patient from a neutral position, and α corresponds to the intervertebral flexion angle, or in other words to the distance between the two screws 10 and 20.

The outer springs 75 and 85 are prestressed, i.e. there is initially a zone I in which the patient exerts a prior force in order to overcome the prestress. In other words, so long as the patient does not exert a threshold force, referenced F₀, the patient does not manage to “overcome” the prestress, and therefore achieves no intervertebral flexion, i.e. the value of the angle α remains zero.

Thereafter, when the patient exerts a force greater than the threshold force F₀, the value of the flexion angle increases linearly with the force exerted by the patient, following the stiffness characteristic of the spring (zone II). This angle α then increases up to a value referenced αₘₐₓ, which corresponds to a value Fₘₐₓ, i.e. the maximum physiological force that the patient can apply. In the context of arthrodesis, to which the present invention applies, this value α is small, as explained above.

This type of curve thus serves to explain the distinction that exists between the concepts of stiffness and of prestress. Thus, if there is no prestress, the force-displacement curve would correspond to a straight line segment extending from the origin. In other words, the slightest force exerted by the patient would tend to push away the screw against the springs 75 and 85. This would not be very advantageous insofar as such a situation would lead to overall instability of the system.

In contrast, the higher the prestress, the greater the value of the threshold force F₀ before any actual movement of the patient occurs. On the graph, dashed lines show an embodiment in which the springs 75 and 85 present the same stiffness, but in association with greater prestress. The threshold force F₀, is then higher, and the maximum angle αₘₐₓ, is smaller.

Furthermore, if the springs 75 and 85 present stiffness that is different, that has an influence on the slope of the straight line extending from the point F₀. Thus, for the same prestress, a chain-dotted line shows an arrangement in which the springs 75 and 85 are stiffer. In other words, the maximum angle α_{max″} that the patient can reach by exerting the maximum physiological force if smaller than the maximum angle αₘₐₓ.

It should be observed that it is possible to adjust the value of the prestress by a small modification to the embodiment of FIG. 4. Thus, if the abutments 31 and 41 are replaced by nuts that are mounted on threaded ends of the rods 30 and 40, it becomes possible to modify the axial positions of the nuts, by screwing them one way or the other. This action is then accompanied by a corresponding variation in the prestress that is imparted to the springs 75 and 85.

During intervertebral extension, co-operation of the branches 53 and 73 together with the screw 10 takes place in a manner analogous to that shown in FIG. 3. In contrast, the screw 20 pushes back the movable branch 83 against the springs 74 and 84 while the additional springs 75 and 85 tend to keep the other movable branch 63′ in contact with the shank 22. In other words, there is no longer any empty space between the branch 63′ and the shank 22 in FIG. 6, unlike that which is shown in FIG. 3.

FIG. 7 shows an additional variant embodiment of the invention. In this figure, mechanical elements that are different from those of FIG. 4 are given the same reference numbers, together with the “prime” sign.

This third embodiment differs from the above-described embodiment in that the jaw 50′ is now movable, and no longer stationary as is the jaw 50 in FIGS. 1 and 4. Thus, the jaw 50′ is mounted to slide on the slideways 30 and 40 against springs 76 and 86 interposed between the tabs 51′ of said jaw and end abutments 32 and 42.

The screws 10 and 20 in this embodiment are mounted in the eyelets defined by the various jaws in a manner analogous to that described above with reference to the second embodiment. Once insertion achieved, two force equilibriums are established, firstly between the screw 10, the springs 74 and 84, and also the springs 76 and 86, and secondly between the screw 20, the springs 74 and 84, and the springs 75 and 85. These force equilibriums are analogous to the equilibrium described with reference to the second embodiment between the screw 20 and the inner and outer springs.

During intervertebral flexion, operation is symmetrical, i.e. the co-operation of the two screws with the four jaws takes place in a manner analogous to that described with reference to the right-hand side of FIG. 5 for the screw 20 and the two movable jaws 60′ and 80. This is shown in FIG. 8, where there can be seen in particular the branch 53′ and the movable jaw 50.

Furthermore, during extension, operation is likewise symmetrical, i.e. the co-operation between the two screws and the four jaws takes place in a manner analogous to that described on the right in FIG. 6, with reference to the screw 20 and the two movable jaws 60′ and 80. Such intervertebral extension is shown in FIG. 9.

FIG. 12 shows an additional variant embodiment of the invention. In FIG. 12, mechanical elements that are analogous to those of FIG. 1 are given the same reference numbers, plus 100.

This embodiment of FIG. 12 differs from that of FIG. 1 in that the two movable jaws 70 and 80 and the two springs 74 and 84 are replaced by a single mechanical member, specifically an extrusion 165. This spring comprises a flexible body 174, e.g. made of an elastomer material, together with two rigid endpieces 170 and 180. Each endpiece is also provided with a stud 173, 183 of generally spherical shape suitable for co-operating with the facing wall of a corresponding shank 112 or 122.

Furthermore, in the embodiment of FIG. 12, the stationary jaws 150 and 160 are formed integrally with the slideway bodies 130 and 140. In other words, these bodies are curved so as to form two ends that connect the slideways together, thereby constituting the jaws 150 and 160. Advantageously, the two slideways and the two jaws are thus formed using a single wire-like element shaped so as to form a loop.

Furthermore, and advantageously, the distance between the slideways 130 and 140 along an axis perpendicular to their main axis, is close to the cross-section of the screw. This enables the slideways to perform a guide function for the screws, so as to keep them well positioned for continuous co-operation with the studs and the curved ends of the slideway. In the above-described embodiments, the screws present cylinders of constant section. However, in FIG. 12, the screws 110 and 120 present constrictions 110′ and 120′ of smaller transverse dimension, co-operating with the two slideways to provide the above-explained guidance.

In the embodiment of FIG. 12, the stationary jaws prevent any intervertebral flexion. In contrast, during extension, the screws compress the flexible body 174, which thus performs the role of the springs 74 and 84. In addition, the endpieces 170 and 180 may be considered as constituting the movable jaws 70 and 80, and the studs 173 and 183 may be considered as constituting the branches 73 and 83. In this respect, it should be observed that the connection between each stud and the corresponding shank is of the point type, thereby making articulation possible, as described above with reference to the first embodiment. In FIG. 12, the two end jaws 150 and 160 are stationary, as in FIG. 1. Nevertheless, provision may be made for at least one of the jaws to be movable, as in FIG. 4 or 7, against at least a pair of springs or at least an additional extrusion.

Like the embodiments of FIGS. 4 and 7 making use of “outer” springs 75, 76, 85, and 86, it is possible to replace those springs with one or two extrusions analogous to the extrusion 165. Under such conditions, the flexible body of each extrusion exerts a function analogous to that of a pair of springs, and its rigid endpiece presents a function similar to that of the movable jaws 53′ or 63′.

Under such circumstances, the flexible body is associated with a stiffness value, and with a prestress, as are the springs. Furthermore, it is possible to surround the flexible body with a rigid chamber, thereby putting a limit on deformation. Under such conditions, the force-displacement curve no longer has two zones as described above, but three zones as shown in FIG. 11.

Firstly there are the two zones I and II analogous to those of FIG. 10, corresponding respectively to the prior force exerted by the patient to overcome the prestress, and then to linear variation of angular movement as a function of force. Finally, there is a zone III of asymptotic shape that corresponds to the flexible body coming into abutment against the walls of the rigid chamber.

In the above examples, stabilization assemblies are shown that connect together only two stages of vertebrae. Nevertheless, provision may be made in accordance with the invention to connect together at least three stages of vertebrae. For this purpose, two main variants may be envisaged as described below.

Thus, firstly it is possible to associate a plurality of extra-discal assemblies analogous to either of the above-described assemblies, as shown in FIG. 13. FIG. 13 shows three pedicular screws 10₁, 10₂, and 10₃, together with a first extra-discal assembly I connecting together a first pair of vertebrae 10₁ and 10₂, and a second stabilization assembly II connecting together the other pair of adjacent vertebrae 10₂ and 10₃. In FIG. 13, as in FIG. 15 described below, each assembly is shown in side view and in highly diagrammatic manner.

Furthermore, and in advantageous manner, provision is made to articulate the two assemblies I and II relative to each other. For this purpose, it is possible to associate a “bead” type spherical separator member P therewith. This member may in particular be in accordance with one of those described and claimed in French patent application 07/59227 filed in the name of the same Applicant on Nov. 22, 2007, the content of which is incorporated herein by reference. It should also be observed that in FIG. 13, the slideways of the assemblies I and II are straight, as contrasted with the curved shape that is described below for the following embodiment.

As an alternative, it is possible to envisage connecting together at least three stages of vertebrae via a single stabilization assembly in accordance with the invention, as shown in FIG. 14. In this figure, mechanical elements analogous to those of FIG. 12 are given the same reference numbers, plus 100.

The embodiment of FIG. 14 for three stages of vertebrae differs from the embodiment of FIG. 12 for two stages of vertebrae in that the slideways 230 and 240 are of greater axial size so as to extend close to these three vertebrae. Furthermore, there are two ends jaw 250 and 260 extending past respective shanks 212, 222 of the end pedicular screws 210, 220. Two extrusions 265₁ and 265₂ are also provided, each connecting the intermediate pedicular screw 215 with one or the other of the end pedicular screws 210, 220. As in the embodiment of FIG. 12, each extrusion is provided with two respective endpieces 273₁, 273₂, 283₁, and 283₂, each serving to provide articulation relative to a corresponding pedicular screw.

Other variant embodiments (not shown) may be envisaged starting from the arrangement of FIG. 14. Thus, it is possible to replace at least one of the two extrusions by springs, analogous to those of FIGS. 1, 4, and 7, for example. Furthermore, in FIG. 14, the two end jaws 250 and 260 are stationary as in FIG. 1. Naturally, provision could be made for at least one of these jaws to be movable, as in the embodiment of FIG. 4, or of FIG. 7, against at least one pair of additional springs or indeed against at least one additional extrusion.

In the embodiment of FIG. 14, the two long slideways are straight, as are the shorter slideways of the first embodiments. Nevertheless, and as shown in FIG. 15, provision may be made to make slideways 230′ and 240′ that present shapes that are curved when seen in side view. Under such circumstances, they advantageously have their concave sides facing towards the vertebral column. In addition, for a curved profile such as that shown in FIG. 15, it is advantageous to leave functional clearance between the facing walls of the slideways and the two extrusions, so as to facilitate movement of the extrusions along the slideways.

There follows a description of a method of putting the assembly of FIG. 14 into place. Nevertheless, it is assumed that the screws 210, 215, and 220 are of constant section, i.e. that they do not have constrictions.

Firstly the various pedicular screws 210, 215, and 220 are put into place by placing a tapering end on each of them, e.g. an end of conical shape. Furthermore, the various extrusions are assembled on the slideways. Because of their resilient nature and because of the absence of screws, it should be observed that the various facing studs come mutually into contact.

Then, the assembly formed by the slideways and the extrusions is fitted onto the screws having their pointed ends. These ends are then inserted between the resilient extrusions, and they then push them back so as to create prestress. Finally, once the assembly is in the position shown in FIG. 14, the various tapering ends are removed and advantageously replaced by an element analogous to the element P shown in FIG. 13.

In this embodiment, the bead P serves to prevent the jaws from sliding along the shanks of the screws, away from the vertebral bodies. Advantageously, an additional bead is provided in the vicinity of the vertebral bodies, so as to prevent sliding towards them. Under such circumstances, the beads are inserted along the shanks of the screws, before putting the slideways and the extrusions into place.

FIGS. 16 to 19 show an additional embodiment of the invention. In these figures, mechanical elements analogous to those of FIG. 12 are given the same reference numbers plus 200.

There can be seen an extrusion 365 comprising a flexible cylindrical body 374 between rigid endpieces 370 and 380. Four slideways are also provided, comprising two first slideways 330₁ and 330₂ forming part of a bent metal wire 330 that forms a loop 350 for passing a first pedicular screw 310. The other two slideways 340₁ and 340₂ forming part of the other wire 340 that is likewise curved form a loop 360 for passing the other pedicular screw 320. Each of these loops defines a jaw in the meaning of the invention.

Each screw presents a middle zone of smaller diameter, like an hourglass. This middle zone co-operates in articulated manner both with a corresponding jaw 350 or 360, and with a facing stud 373 or 383, as in the above-described embodiments.

The first wire 330 is slidably mounted in orifices 370′ formed in the first endpiece 370 adjacent to the loop 350 and then also extends, likewise in slidable manner, in openings 374′ formed in the flexible body. Finally, the ends of this wire are fastened to the opposite endpiece 380, by any appropriate means. In a variant, provision can be made for these ends to pass through the opposite endpiece in slidable manner and also to be provided with abutment means serving to retain the endpiece. In other words, the endpiece is constrained to move with the screw 310 in translation in the direction of compressing the flexible body, i.e. when the two endpieces move towards each other.

In analogous manner, the second wire 340 extends slidably successively through orifices 380′ formed in the endpiece 380 that is adjacent to the loop 360, and then through additional openings 374″ formed in the flexible body. As mentioned above with reference to the first wire, the second wire may either be fastened to the endpiece 370 opposite from the loop 360, or it may be slidably mounted relative thereto, being associated with abutment means.

In the event of intervertebral flexion, as shown in FIG. 19, the two pedicular screws are caused to move apart. Under such conditions, the right screw 320 takes the left endpiece 370 to the right, while the left screw 310 takes the right endpiece 380 to the left, thereby moving the two endpieces towards each other. Under such conditions, the flexible body 374 is compressed, such that it performs a damping function and tends to return the two screws towards each other, back to their original position.

Furthermore, during intervertebral extension (not shown in the figures), each loop tends to move away from the pedicular screw with which it was originally in contact. This movement is accompanied by corresponding compression being applied to the flexible body, which therefore once more performs a damping function and tends to return the assembly to its initial, rest position.

As can be seen from the above, not only intervertebral extension, but also flexion leads to the flexible body being compressed, such that it acts as a damper in both cases. There can thus be seen to be differences between this embodiment of FIG. 16 and the embodiment of FIG. 7, for example. Thus, in FIG. 7, each pair of jaws is associated with two distinct return means, each operating in a respective direction. In contrast, in the embodiment of FIG. 16, these two return means comprise single return means 374, handling both opposite directions of movement.

It can be seen that the more flexible the body the greater the movement it allows between the two vertebrae. Thus, with arthrodesis, it is appropriate to select a flexible body that is relatively rigid, so as to limit intervertebral movement. It is also possible to make provision for associating the flexible body with a deformation limit. Thus, by way of example, the flexible body 374 may be surrounded by means of a rigid cylindrical chamber 390 that is bonded to one or other of the endpieces, as shown in FIG. 20.

With reference to FIG. 21 in which the flexible body is in the rest position, its walls extend at a distance from the walls of the chamber. Under such conditions, and at a certain state of deformation, the walls of the flexible body come into contact with the walls of the chamber, thereby preventing any additional movement (FIG. 22).

As an alternative, the flexible body 374 may be surrounded by means of at least one rigid ring 392 (FIGS. 23 and 24) that extends over a substantial portion of the flexible body. It should be observed that unlike creating a compression chamber as described in the preceding paragraph, there is no empty space between the walls of the ring and the flexible body when the flexible body is in its rest position. In other words, in the contact zone between the ring and the flexible body, the flexible body cannot deform. Under such conditions, the deformable zone of the flexible body corresponds solely to its portion that is not surrounded by the ring (see FIG. 25), i.e. two segments Z₁ and Z₂.

In both of the preceding embodiments, either with the chamber 390 or with the ring 392, deformation of the flexible body 374 is limited, thereby limiting the relative movement between the two pedicular screws. Nevertheless, other means may be provided for limiting this stroke. In non-limiting manner (not shown), mention may be made for example of any appropriate abutment means serving to stop the stroke of the slideways relative to the rigid endpieces.

The implementation of the device shown in FIGS. 16 et seq., when associated with a chamber as shown in FIGS. 20 to 22, can be seen more particularly in FIG. 26, which is analogous to FIG. 10. There can thus be seen a curve repenting the variations or movements between two vertebrae as a function of the force F exerted on the flexible body 374. The origin of the curve corresponds to a rest position, i.e. a position in which there is no stress on the flexible body, when the screws are not inserted between the studs and corresponding jaws. Thereafter, when the screw is put into place this leads to a certain amount of compression force on the flexible body, which force comes into equilibrium with a certain amount of movement between the screws. This corresponds to the point (F₀, α₀) on the curve.

Thereafter, when the patient leans either forwards or backwards, as explained above, this leads in both situations to the flexible body being compressed, thereby opposing such movement. In addition, the presence of the chamber puts a limit on the movement, as represented by the asymptote A corresponding to an intervertebral angle of inclination αₘₐₓ. The curve plotting force as a function of movement is thus limited between firstly a “relative” origin as constituted by the points F₀ and α₀, and secondly the asymptote A.

In the embodiment of FIG. 16 et seq., the return member is a solid flexible body. Nevertheless, in a variant, provision may be made to use a coil spring extending along the axis between the two screws. The two free ends of the spring are then fastened by any appropriate means against the respective rigid endpieces. This embodiment is advantageous insofar as it enables friction to be reduced in operation, particularly compared with the friction associated with the rods moving in the orifices in the flexible body.

In the various embodiments described above, the extra-discal assembly in accordance with the invention has at least two vertebral screws. Under such conditions, the various screws are generally implanted on one side of the vertebral column, at a distance from the middle vertebral axis thereof, with reference to the patient standing. It is also possible to provide for implanting two sets of vertebral screws, on both sides of this middle axis, with each set of screws then forming part of a corresponding extra-discal assembly.

Nevertheless, in a variant, provision may be made to use not vertebral screws, but rather rods, each forming part of a connection element, e.g. connecting together two pedicular screws at the same vertebral stage. Under such conditions, the extra-discal assembly in accordance with the invention has at least two such rods placed one above the other substantially along the middle vertebral axis, together with jaws, slideways, and return means, as in the above-described embodiments.

1.-19. (canceled)

20. An extra-discal assembly for intervertebral stabilization for arthrodesis, the assembly comprising:
at least two rods referred to as vertebral rods, suitable for co-operating with at least two different vertebrae;
at least two pairs of jaws, each pair of jaws being placed in the vicinity of a corresponding rod;
at least two slideways defining a longitudinal axis of said assembly, at least one jaw in each pair of jaws being suitable for sliding along at least two slideways, along their respective longitudinal axes; and
return means for returning at least one jaw in each pair of jaws towards a corresponding vertebral rod;
each vertebral rod being suitable, in operation, for moving at least one jaw along the slideways against the return means, each vertebral rod also being articulated relative to each jaw.

21. An assembly according to claim 20, wherein said articulation between the rod and each jaw acts via a single contact point between said rod and the facing wall of each jaw.

22. An assembly according to claim 21, wherein at least one jaw comprises a curved connection branch having a radius of curvature greater than the radius of curvature of the rod.

23. An assembly according to claim 22, wherein the curved branch is secured to two tabs mounted on the two slideways, optionally in slidable manner.

24. An assembly according to claim 22, wherein the return means comprise two springs, each of which is mounted on a corresponding slideway, these two springs being suitable for returning a curved branch towards the vertebral rod.

25. An assembly according to claim 23, wherein at least one jaw includes an articulation stud engaged against a corresponding vertebral rod.

26. An assembly according to claim 25, wherein the return means comprise a solid flexible body and the stud forms part of a rigid endpiece secured to the flexible body.

27. An assembly according to claim 20, wherein at least one pair of jaws comprises a first jaw slidable along the slideways against the return means, together with a second jaw mounted stationary on the slideways.

28. An assembly according to claim 20, wherein at least one pair of jaws comprises first and second jaws mounted slidable along the slideways, with the return means comprising first and second return means, each movable jaw being suitable for sliding against corresponding return means.

29. An assembly according to claim 20, wherein the first and second return means are merged into a single return means comprising a central return member between two endpieces each defining a first respective jaw, in that two pairs of slideways are provided, each pair defining a respective second jaw, and in that each pair of slideways is suitable for sliding relative to the endpiece adjacent to the jaws defined by said slideways, said pair of slideways in contrast being secured to move in translation with the opposite endpiece, at least in the direction in which the two endpieces approach each other.

30. An assembly according to claim 20, wherein the central return member is a helical spring.

31. An assembly according to claim 20, wherein each rod forms part of a corresponding vertebral screw, in particular a pedicular screw.

32. An assembly according to claim 20, wherein each screw is secured to a connection element suitable for connecting together two vertebral screws for implanting in a common vertebral stage.

33. An assembly according to claim 20, wherein the assembly includes stroke-limitation means for limiting the stroke between two adjacent rods.

34. An assembly according to claim 20, wherein the stroke-limitation means are means for limiting the compression of the deformable body, forming part of the return means.

35. An assembly according to claim 20, wherein the compression-limitation means comprise a rigid chamber extending at a distance from walls of the deformable body (374) when the deformable body is in a rest position.

36. An assembly according to claim 34, wherein the compression-limitation means comprise at least one ring surrounding a portion of the deformable body so as to limit its deformation zone.

37. An assembly according to claim 20, wherein it includes means serving to prevent the slideways and the jaws sliding along the rods, along the main axes thereof, in at least one direction.

38. An assembly according to claim 20, wherein two slideways are suitable for guiding the rods, in particular by co-operating with respective zones of said rods presenting a cross-section that corresponds substantially to the distance between the slideways.

39. A method for fixing an assembly according to claim 20, wherein:
each rod is implanted in a vertebrae;
the rods are moved manually towards each other;
the movable jaws are moved axially towards the rods so as to insert each rod through an eyelet defined by the corresponding pair of jaws;
the external action exerted on the rods is released so that each rod comes to bear against one of the jaws of each pair of jaws.
